(19) United States
(12) Patent Application Publication
(10) Pub. No.: US 2025/0018103 A1
(43) Pub. Date: Jan. 16, 2025

(54) COOLING DEVICE FOR A MEDICAL TREATMENT DEVICE

(71) Applicant: Xenios AG, Heilbronn (DE)

(72) Inventors: Heiko Giebler, Mössingen-Öschingen (DE); Nektarios Panagias, Reutlingen (DE); Patrick Fernes, Reutlingen (DE)

(21) Appl. No.: 18/712,906

(22) PCT Filed: Nov. 23, 2022
(86) PCT No.: PCT/EP2022/083037

§ 371 Date: May 23, 2024

(30) Foreign Application Priority Data
Nov. 24, 2021 (DE) 10 2021 005 829.1

(51) Int. Cl. A61M 1/36

(52) U.S. Cl. CPC A61M 1/3623; A61M 2205/3606

(57) ABSTRACT

The present disclosure relates to cooling devices for medical treatment devices and to corresponding medical treatment devices. Accordingly, a cooling device includes a housing having an air inlet, an air outlet, and a housing interior. An air channel is provided within the housing interior, wherein a wall of the air channel defines a continuous inner cavity fluidly connected to the air inlet and the air outlet. The inner cavity, the air inlet, and the air outlet are hermetically sealed from the interior of the housing by the air channel. The air channel includes at least one fan arranged in the inner cavity and adapted to provide an air flow from the air inlet to the air outlet. The cooling device includes at least one fan arranged in the housing interior and arranged to provide an air flow flowing at least partially around the air channel.

### CROSS-REFERENCE TO RELATED APPLICATIONS

The present application is the national stage entry of International Patent Application No. PCT/EP2022/083037, filed on Nov. 23, 2022, and claims priority to Application No. DE102021005829.1, filed in the Federal Republic of Germany on November 24, the disclosures of which are expressly incorporated herein in their entirety by reference thereto.

### TECHNICAL FIELD

The present disclosure relates to cooling devices for medical treatment devices and to corresponding medical treatment devices, for example extracorporeal circulatory support devices including such a cooling device.

### BACKGROUND

Medical treatment devices, such as extracorporeal circulatory support devices or blood treatment devices, require a precise control and/or regulation in order to provide a therapy required for a patient and which is adapted to the pathophysiological conditions. For example, extracorporeal circulatory support systems or blood treatment devices, such as membrane oxygenators, generally have one or more pumps that must be actuated in an accordingly timed manner. Furthermore, for a synchronization of such pumps with the heart cycles or heart actions of the patient, an evaluation unit may be provided, which detects or receives ECG signals of the patient in order to subsequently utilize them for providing a trigger signal. For example, such an evaluation unit can be provided in the form of an ECG card or an ECG module of the treatment device.

Electronic components are provided for the control/regulation, which may be present in the form of modules, for example. The electronic components generate heat, which can be considerable depending on the complexity of the control/regulation or operating mode of the treatment device. To avoid possible damage to such sensitive components due to overheating, the generated residual heat should be removed or dissipated such that the electronic components are operated within a predefined temperature range.

The electronic components are usually located in the housing interior of the respective treatment device. This is advantageous from the point of view of mechanical and structural stability as well as hygiene. This is because in this way the components are largely protected in the event of an accidental impact and the outer surface of the treatment device can be easily cleaned, for example, between therapy applications.

However, it is problematic that the waste heat generated by the electronic components accumulates in the interior of the treatment device, which may cause an overheating of the electronic components. Furthermore, this may result in an increase of the temperature of the surface of the treatment device. To prevent overheating of the electronic components and to avoid high temperatures or even burns of the operating personnel when touching the surface as far as possible, an active cooling is required. In the prior art, cooling systems are known which use a fresh air supply flowing in from the outside to actively cool the inside of the housing, in which the operated electronic components are housed, with ambient air. Alternatively, heat sinks may be provided on the surface of the treatment device or housing to provide a passive cooling with ambient air.

However, introducing ambient air into the interior of the device poses the risk of an uncontrolled introduction of dust, liquids, and humidity into the interior of the device. Such contamination renders it more difficult to perform the cleaning measures required to comply with the relevant hygiene standards. In addition, such contamination may result in defects in the electronic components, such that shorter maintenance intervals become required, which shortens the operational time of the respective treatment device. Heat sinks on the surface of the device, which also provide an inefficient cleaning, further complicate the handling of the treatment device and require inappropriate sizing and dimensions.

Accordingly, there is a need to improve the removal of accumulated heat within a medical treatment device and to simplify compliance with hygiene standards or reduce the corresponding cleaning effort.

### SUMMARY

Based on the known state of the art, it is an aspect of the present disclosure to enable an improved removal of accumulated waste heat from the interior of a treatment device.

Accordingly, a cooling device for a medical treatment device is suggested, which includes a housing having an air inlet, an air outlet and a housing interior defined by a housing wall. An air channel is arranged within the housing interior, wherein a wall of the air channel defines a continuous inner cavity fluidly connected to the air inlet and the air outlet. The inner cavity, the air inlet, and the air outlet are hermetically sealed from the housing interior by the air channel. The air channel includes at least one fan arranged in the inner cavity and adapted to provide an air flow from the air inlet to the air outlet. Furthermore, the cooling device includes at least one fan arranged in the housing interior and arranged to provide an air flow at least partially flowing around the air channel.

Due to the hermetic sealing, ambient air can only enter the inner cavity. This is because the air inlet, the air outlet and the inner cavity are all hermetically sealed from the interior of the housing. In other words, ambient air cannot enter the interior of the housing via the air inlet. Rather, according to the disclosure, the ambient air is directed or fed directly into the inner cavity. Although the inner cavity extends from the air inlet to the air outlet in the interior of the housing, it does not have any openings to the interior of the housing, such that the ambient air exits the inner cavity via the air outlet. In this manner, it is prevented that, for example, dust, dirt, moisture or other contaminants can enter the interior of the housing. Any electronic components provided inside the housing are thus protected, e.g., dust and water.

Both the air inlet and the air outlet can be adapted to the dimensions of a connecting section at the respective portion of the air channel. A hermetic seal of the air inlet and the air outlet to the interior of the housing may be provided, for example, by means of a corresponding sealing ring or sealing means. Alternatively, the air channel or the corresponding wall sections at the portion of the air inlet and at the portion of the air outlet and at least the corresponding housing sections in which the air inlet and the air outlet are located may be formed integrally or from a single part. In some embodiments, however, the air channel is detachably connected to the housing so that the air channel may be easily decoupled and removed for a cleaning and/or disinfection process.

While the interior of the housing is separated from the ambient air, effective cooling of the interior of the housing is enabled by the air flow provided in the air channel. This is because at least one fan is provided in the interior of the housing, which provides an air flow that at least partially flows around the air channel. The air flow in the interior of the housing is thus fluidically decoupled from the ambient air and the air flow provided in the air channel, but allows waste heat or residual heat formed and accumulated in the interior of the housing to be effectively dissipated or conducted via the wall of the air channel and absorbed and dissipated or removed by the air flow in the air channel. In other words, the wall of the air channel, which is typically formed of a thermally conductive material, can be cooled by the ambient air and the correspondingly provided air flow, and waste heat can be absorbed by this air flow via a surface of the wall facing the interior of the housing. The fan inside the housing causes the air inside the housing to be circulated, on the one hand, in order to absorb waste heat from electronic components from various portions inside the housing, and, on the other hand, causes that the air channel is at least partially surrounded by this air or internal air in order to dissipate the waste heat accordingly via the wall of the air channel.

For example, the cooling device includes no heat sink within the housing interior other than the air channel. In this embodiment, no further heat sink is arranged adjacent to the air channel or is thermally connected to the air channel.

Accordingly, a separation of the housing interior from the ambient air and external influences is provided and components present in the housing interior are protected. Nevertheless, a high efficiency of cooling is provided due to the circulation of the internal air and the active heat dissipation or transfer by means of the air flow in the air channel. Thus, direct cooling of the interior of the housing with ambient air and passive cooling by means of external heat sinks can be dispensed with. Conventional filters, which are usually provided for the ambient air towards the housing interior, can thus also be dispensed with. This also considerably simplifies maintenance of the cooling device and the treatment unit.

The waste heat may be generated directly in the housing, for example, if the housing is a housing of a medical treatment device to be cooled and the electronic components are thus at least partially located inside the housing or are directly thermally coupled thereto. Alternatively, however, the housing may also be formed as an insert or module, which may be inserted into or integrated into a housing of a medical treatment device to be cooled and can be thermally coupled thereto, for example, by means of corresponding sections of the housing.

By dissipating or removal of the waste heat, in addition to cooling the interior of the housing, it is also possible to reduce the temperature at an external surface of a medical treatment device to be cooled. Consequently, in this manner, both the operating temperatures of electronic components can be kept low and contacts of operating personnel with potentially dangerous surface temperatures may be prevented.

For example, the at least one fan of the housing interior is spaced apart from the wall of the air channel and/or the at least one fan of the housing interior and the air channel are arranged at opposing end portions of the housing. In an embodiment, the at least one fan of the housing interior is accordingly spaced apart from the wall of the air channel. In this embodiment, e.g., also no liquid-based thermal coupling is present between the air channel or wall of the air channel, respectively, and the housing interior, e.g., via a coolant. For example, the cooling device also includes no heat sink within the housing interior other than the air channel. Accordingly, no impingement cooling may be provided on such further heat sink within the housing interior by means of convection of the air flow.

The separate arrangement or spacing of the fan from the wall allows the provided air flow to pass through a corresponding portion of the housing interior. This allows the corresponding waste heat, which may accumulate at different areas or portions inside the housing, to be dissipated more effectively. Furthermore, attachments to the wall of the air channel can thus be avoided, such that, for example, an outer side of the wall may also be optimized for dissipation and a mechanical load on the air channel may be reduced.

In some embodiments, the wall of the air channel is spaced apart from the housing wall in the portion or region between the air inlet and the air outlet. The portion may correspond substantially to the complete portion or region between the air inlet and the air outlet, such that the air channel or its wall is completely spaced apart from the housing wall except for the connecting sections. In this way, an even larger surface is provided for heat transfer. However, the wall of the air channel may be supported by one or more fastening means providing attachment to the housing wall.

In this regard, the at least one fan of the housing interior may be arranged such that the air flow provided in the housing interior flows substantially circumferentially around the wall of the air channel. For example, the flow hence not only occurs in longitudinal direction of the air channel, but also in a circumferential direction.

For example, the air flow in the housing interior may be substantially or at least partially transverse and/or perpendicular to the air flow in the air channel, wherein the at least one fan of the housing interior and the air channel can be arranged at opposing end portions of the housing. The at least one fan of the housing interior may be oriented towards the air channel or towards the inner side of the housing wall. Accordingly, the provided air flow can be directed at least partially along the housing wall, such that the transfer or dissipation of waste heat may be further improved and/or may be provided directly for heat generating components. By circulating the air flow around the wall, the duration and thus the efficiency of the heat transfer is further improved.

Furthermore, multiple fans can be arranged in the housing interior, e.g., two fans, which are arranged adjacent to each other and can provide an air flow in substantially opposing directions. This may provide an increased circulation within the housing and the interior air is flowed onto or around an increased possible area or portion of the housing.

As described above, the interior of the housing is protected from contaminants due to the hermetic sealing while still allowing an active cooling of the interior of the housing due to the air flow in the air channel. Based on the flow rate or the volume flow in the air channel and the ambient air temperature on the one hand and the generated and existing waste heat as well as the air flow in the housing interior on the other hand, the air temperature exiting at the air outlet may be accordingly increased. However, at typical ambient temperatures and operating conditions of the medical treatment device to be cooled, the air temperature may be sufficiently low for further cooling capacities. Accordingly, the air channel at the end or portion of the air outlet may be shaped or formed such that the air flow at the air outlet is at least partially inclined to the air flow between the air inlet and the air outlet.

Thereby, the air flow may flow towards or around one or more external components of the treatment device due to the inclination, thus providing additional impingement cooling or film cooling at the air outlet. In some embodiments, the external components are mechanical components and may include, e.g., a pump drive that generates corresponding heat in the operating mode. In some embodiments, however, the external components do not include electronic components.

It is not necessary that the slope or inclination is present for the entire air flow. In other words, a portion of the air flow may exit the air outlet without an angle of inclination or at a different angle of inclination. Thus, several spaced apart or separate external components can be cooled by the expansion of the air flow. In some embodiments, the exiting air flow is fully inclined to the air flow in the inner cavity. Thereby, one or more external components of the treatment device, which for example generate a larger amount of waste heat, may be selectively cooled.

For example, the angle of inclination may be 10 degrees to 80 degrees. For example, an external component of the treatment device may thus be cooled at different portions and in different ways. For example, the external component may be attached to an end portion of the housing, wherein a portion of the generated waste heat may be reduced or dissipated by means of the internal air at this end region. The angle of inclination allows a portion of this component, which is not in this end region and is spaced apart therefrom, to be further cooled by impingement or impingement cooling with the exiting air flow due to the inclination.

In order to further vary or accelerate the air flow in the air channel without causing unfavorable turbulence, two or more fans can be provided in the air channel, wherein the fans are arranged at the portion of the air inlet and are equally spaced apart in the longitudinal direction of the air channel with respect to the air inlet.

The longitudinal direction of the air channel is generally to be understood as the extension of the air channel or its wall in the direction of flow or a direction which is specified by a main orientation of the air channel between the air inlet and the air outlet. The longitudinal direction can thus be a direction which is greater than any extension of the air channel in the cross-section of the air channel. By spacing the fans equally, they can thereby be aligned in parallel to each other and in a direction perpendicular to the longitudinal direction.

Furthermore, a corresponding opening may be provided in the air channel for each fan. In some embodiments, the air channel includes two openings (and two corresponding fans) at the portion or end of the air inlet and one opening at the end or portion of the air outlet, wherein the opening at the air outlet can correspond to the dimensioning of the air outlet. Due to the arrangement, the fans may be fluidically connected to each other, but can provide (two) substantially separate partial air flows, which are (at the latest) combined at the air outlet.

The wall of the air channel, which is typically formed of a (particularly) thermally conductive material, can assume different shapes and, for example, have a substantially continuous form to define a corresponding air channel or inner cavity. Thus, the air channel can have a cross-section that is, for example, round, ellipsoidal, trapezoidal or even rectangular. In order to further improve the heat transfer or the dissipation of the waste heat, the air channel can have a plurality of inner heat exchange elements in the inner cavity, which extend substantially in the longitudinal direction of the air channel and can protrude from the wall into the inner cavity.

The inner heat exchange elements may be arranged substantially in parallel to each other. In some embodiments, the inner heat exchange elements extend substantially from the air inlet to the air outlet. The inner heat exchange elements may also be (slightly) curved or sinusoidal in shape and/or include a plurality of heat exchange elements that are spaced apart from each other in the longitudinal direction. The inner heat exchange elements increase a surface area for dissipating or transferring waste heat to the air flow provided in the air channel, allowing improved cooling of the interior of the housing. The internal heat exchange elements may be ribbed and/or planar or sheet-like in shape, so as to provide an increased possible surface area.

In some embodiments, the inner heat exchange elements are arranged on opposing sides of the wall in the cross-section of the air channel and the heat exchange elements extending towards each other are arranged offset from each other in the circumferential direction of the air channel.

This provides that there is a gap between adjacent heat exchange elements and that the heat exchange elements do not overlap in any direction perpendicular to the longitudinal direction or that the heat exchange elements do not adjoin each other. In other words, some interlocking of the heat exchange elements is provided without the heat exchange elements engaging each other because of this. The surface area provided for dissipating the waste heat is thereby further improved without the air flow in the air channel being significantly impeded by the heat exchange elements.

To further improve the dissipation or transfer of waste heat, the air channel may—alternatively or additionally—have a plurality of outer heat exchange elements on the wall of the air channel, which protrude into the housing interior. In some embodiments, the outer heat exchange elements extend circumferentially on the wall and are longitudinally spaced from each other. The outer heat exchange elements thus enable improved absorption of waste heat and conductance or transfer to the inner cavity, where the waste heat is effectively dissipated by means of the provided air flow.

To provide a large possible surface area, the outer heat exchange elements can extend continuously or continuously in the circumferential direction. Optionally, however, an offset or spacing can also be provided in the circumferential direction, for example, to specifically influence or direct the air flow. In this manner, turbulence can be provided, for example, which can extend the duration of the heat transport. With regard to the air flow within the air channel and in the longitudinal direction of the air channel, the extension in the circumferential direction has the effect that the efficiency in dissipating the waste heat can be further improved.

Optionally, the outer heat exchange elements may also extend longitudinally as long as they do not overlap longitudinally with adjacent outer heat exchange elements. The outer heat exchange elements, like the inner heat exchange elements, may be, for example, ribbed or planar or sheet-like in shape.

The cooling device may further include a nozzle configured to direct and/or accelerate the air flow provided in the inner cavity at the air outlet. By means of the nozzle, a targeted cooling of external components can be achieved, for example of a pump drive of a medical treatment device to be cooled. The nozzle may thus not only predefine a specific orientation of the exiting air flow, but also a flow velocity based on appropriate dimensioning. This may, for example, provide a targeted and precise impingement cooling of an external component.

In some embodiments, the nozzle is tiltable relative to the housing wall and/or a diameter of the nozzle is variable or adjustable.

The inclination and the diameter may be manually adjustable and/or be adjusted by means of a corresponding control and/or regulating unit. The inclination can be relative to the air channel, such that the air flow may also be deflected in this portion or at the portion or end of the air outlet in addition to any potentially present adapted geometry of the air channel at portion or end of the air outlet of the air channel. The inclination and/or the diameter may be adapted to the arrangement and/or the requirements or the generated waste heat of the external components.

Alternatively, the nozzle may be formed as part of the portion or end of the air outlet of the air channel and thus predefine an angle of inclination of the air flow at the air outlet at least partially relative to the air flow between the air inlet and the air outlet.

In some embodiments, the cooling device further includes a control and/or regulating unit or is communicatively connectable to a control and/or regulating unit of a medical treatment device to be cooled. The inclination and/or diameter may be adjusted based on a detected operating state or condition of the treatment device and/or a component or of a functional unit of the treatment device by means of the control and/or regulating unit to provide the desired or preferred orientation and/or a required (minimum) volume flow.

The cooling device may further include a control and/or regulating unit adapted to receive a temperature measurement of the interior of the housing, the air channel, and/or a medical device cooled by the cooling device and/or an operating condition or state of a medical device cooled by the cooling device, and to control/regulate the air flow, which is provided by the at least one fan in the air channel, based on the temperature measurement and/or the operating condition.

In this manner, the air flow may be adjusted based on a provided waste heat, wherein the waste heat is detected or determined based on the temperature measurement and/or the operating condition. For example, a standby condition may require a low air flow or even no air flow, while normal operation may require a correspondingly higher air flow. Based on the received operating state or a received operating mode and, for example, corresponding characteristic curves, the at least one fan in the air channel may be accordingly controlled/regulated. The control and/or regulation unit may be connectable to an interface of the medical device to receive the operating state, wherein an operating mode may be indicative of the operating state.

A more accurate detection of the generated waste heat may be enabled based on a received temperature measurement. For this purpose, for example, one or more temperature sensors may be integrated in the cooling device or in the interior of the housing and/or in the air channel and be communicatively connected to the control and/or regulating unit.

The operating state or mode or temperature measurement may also be received from at least one specific component of the medical treatment device to be cooled. For example, the control and/or regulating unit may specifically receive a current operating mode of a pump drive or a current pump status and be configured to adjust the air flow accordingly.

The above aspect is furthermore solved by a medical treatment device, e.g., an extracorporeal blood treatment device and/or an extracorporeal circulatory support device, including a cooling device as described above and according to the present disclosure.

For example, the medical treatment device may be formed as a console, e.g., in a portable embodiment. For example, the medical treatment device may be configured as an extracorporeal membrane oxygenator (ECMO) or other oxygen delivery device, an extracorporeal circulatory support (ECLS) device, or an extracorporeal carbon dioxide removal (ECCO2R) device. As described above, the housing of the cooling device may be designed, for example, as an insert or module which is inserted into or integrated into a housing of the medical treatment device and thermally coupled thereto, for example by means of corresponding sections of the housing. In this manner, a hermetic sealing of the sensitive electronic components is ensured, while nevertheless providing sufficient cooling.

In some embodiments, the medical treatment device includes a housing, wherein the housing of the cooling device is or may be formed at least as part of the housing of the medical treatment device. In some embodiments, the housing of the cooling device corresponds to the housing of the medical treatment device.

Thus, a substantially enclosed console may be provided as a medical treatment device, wherein the interior of the housing of the cooling device substantially corresponds to the interior of the housing of the medical treatment device. Electronic components are thereby hermetically sealed and protected, e.g., as only an air channel is provided in which an air flow is provided between an air inlet and an air outlet. However, the air channel is not fluidically connected to the interior of the housing of the medical treatment device. Accordingly, such a closed console is advantageous not only from the point of view of reduced maintenance of the electronic components, but also from a hygienic point of view. Potentially health-endangering germs cannot penetrate into the interior of the housing, and cleaning and disinfection of the treatment device is hence considerably facilitated. Thus, the cooling device according to the present disclosure and the corresponding medical treatment device may also be used in an intensive care unit, since only the outer surface of the treatment device needs to be cleaned and disinfected. Since external heat sinks can further be dispensed with on the surface, the cleaning and disinfection is even further facilitated according to the disclosure.

### BRIEF DESCRIPTION OF THE DRAWINGS

FIG. 1 shows a schematic representation of a cooling device that is integrated in a medical treatment device.

FIG. 2 shows a schematic sectional view of a medical treatment device with a cooling device with a flow profile of the air flow within the housing interior.

FIG. 3 shows a corresponding schematic sectional view of the medical treatment device according to FIG. 2 of the air flow through the air channel.

FIG. 4 shows a perspective sectional view of an embodiment of the air channel of the medical treatment device according to FIG. 2.

FIG. 5 shows a schematic sectional view of the medical treatment device according to FIG. 2 with an advantageous configuration of the air outlet.

FIG. 6 shows a perspective side view of the air channel according to FIG. 4.

FIG. 7 shows a perspective front view of the air channel according to FIG. 6.

FIG. 8 shows a perspective rear view of the air channel according to FIG. 4.

### DETAILED DESCRIPTION

In the following, embodiments will be explained in more detail with reference to the accompanying Figures. In the Figures, corresponding, similar, or like elements are denoted by identical reference numerals and repeated description thereof may be omitted in order to avoid redundancies.

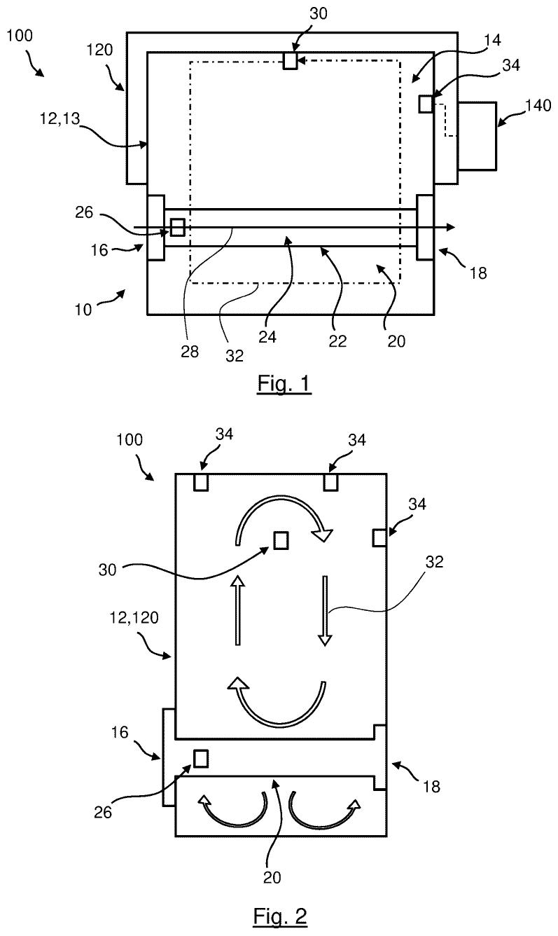

A schematic depiction of a cooling device 10 is shown in FIG. 1. The cooling device 10 is integrated in a medical treatment device 100 or is formed as part of the treatment device 100. Accordingly, a housing 12 of the cooling device 10 also forms part of the housing 120 of the medical treatment device 100. For example, the housing 12 of the cooling device 10 may include the essential electronic components of the medical treatment device 100 in the housing interior 14 defined by the housing wall 13, wherein the housing 120 of the medical treatment device 100 may include further sections which do not correspond to the housing 12 of the cooling device 10 and include, for example, mechanical components required for the treatment or a therapeutic procedure. Alternatively, however, the housing 12 of the cooling device 10 may be formed entirely by the housing 120 of the medical treatment device 100.

The housing 12 of the cooling device 10 includes an air inlet 16 and an air outlet 18 at opposing end portions of the housing 12. An air channel 20 is provided between the air inlet 16 and the air outlet 18, which fluidically connects the air inlet 16 to the air outlet 18 and to the ambient air. The air channel 20 is substantially formed by a wall 22 extending from the air inlet 16 to the air outlet 18 which defines a continuous inner cavity 24. Accordingly, ambient air can enter the inner cavity 24 via the air inlet 16 and the air outlet 18. A fan 26 is furthermore provided in the air channel 20, which is arranged in the inner cavity 24 in close or direct proximity to the air inlet 16. The fan 26 is configured to draw or convey ambient air into the inner cavity 24 via the air inlet 16 and to provide an air flow 28 that flows from the air inlet 16 to the air outlet 18 and exits the air channel 20 at the air outlet 18.

A fan 30 is also arranged in the housing interior 14 at an end portion of the housing 12 opposite the air channel 20. The fan 30 is also adapted to provide an air flow 32. In contrast to the air flow 28, however, no ambient air is drawn in, but only the air present in the housing interior 14 is circulated, as indicated by the dashed arrow. The fan 30 is arranged in such a way that the air inside the housing 14 is circulated and the air flow 32 at least partially surrounds the air channel 20.

An advantageous feature of this embodiment is that the housing interior 14 is hermetically sealed from the outside and thus neither ambient air nor corresponding contaminants can enter the housing interior 14. Accordingly, electronic components 34 located within the housing interior 14 and communicatively coupled to corresponding mechanical components 140 are protected. The hermetic sealing is provided at least in part by the air channel 20, which is connected to the air inlet 16 and the air outlet 18 in such a way that ambient air can only enter the inner cavity 24 and not the housing interior 14. The inner cavity 24 is also hermetically sealed from the housing interior 14, such that the housing interior 14 is decoupled from external influences.

The heat or waste heat generated by the electronic components 34 cannot be dissipated via the ambient air due to the hermetic seal. The air flow 28 provided in the air channel 20 carries fresh ambient air through the air channel 20, which can absorb waste heat from the housing interior 14 via the wall 22. The waste heat is dissipated or transferred to the air flow 28 via the wall 22 due to the circulation and the corresponding air flow 32, which also surrounds the air channel 20. In this manner, waste heat can be effectively removed from the housing interior 14 without allowing ambient air to enter the interior of the housing 14. Furthermore, due to the advantageous arrangement, no additional heat sinks are required on the outer surface of the medical treatment device 100.

Thus, separation of the housing interior 14 from ambient air and external influences is provided, thereby protecting the electronic components 34 located in the housing interior 14. In addition, a high degree of efficiency of cooling or dissipation of waste heat is achieved.

In FIG. 2, flow profiles of the air flow 32 inside the housing interior 14 are shown, and it can be seen that the at least one fan 30 (for example, two or three fans may be provided) provides an advantageous circulation of the interior air, thereby substantially removing or transporting waste heat from the entire housing interior 14 and away from the electronic components 34. Also shown in the Figure is air channel 20, which includes a separate fan 26 and which is connected to air inlet 16 and air outlet 18. It can be seen that, despite the significant circulation and due to the hermetic seal, no ambient air enters the housing interior 14. The housing 12 of the cooling device 10 is optionally formed by the housing 120 of the treatment device 100, as shown herein.

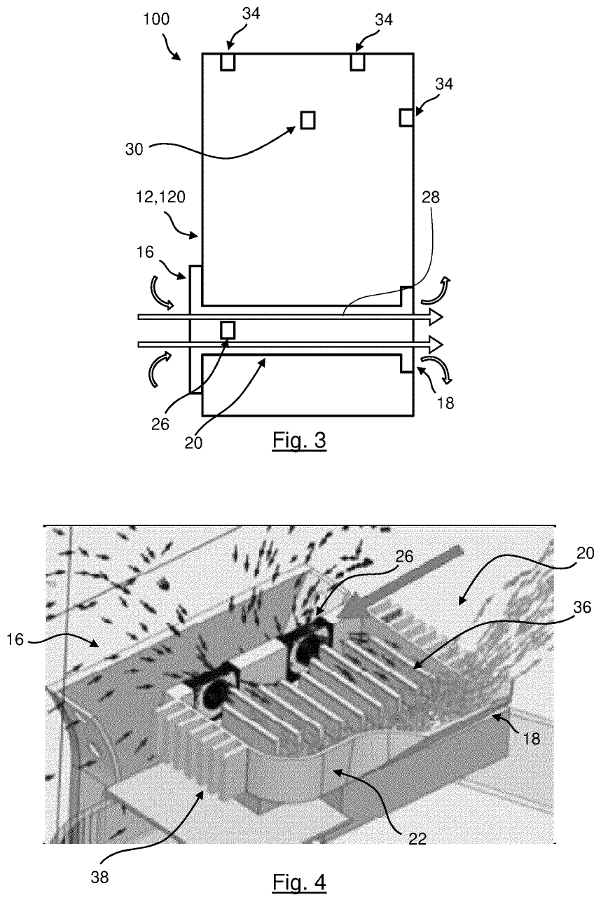

Similarly, it is shown in FIG. 3 that an air inlet 16 is located at the front of the medical treatment device 100 and, although the ambient air may be drawn or conveyed into the air channel 20 via the fan 26 as an air flow 28, the ambient air cannot enter the interior of the housing.

A perspective sectional view of the air channel 20 and the corresponding mode of operation is shown in FIG. 4. In the present example, two fans 26 are provided, which are arranged in parallel and adjacent to each other at the air inlet side or portion. The fans 26 are provided with corresponding openings and cause ambient air to enter the air channel 20 or its inner cavity 24 via the air inlet 16. A plurality of heat exchange elements 36 are provided in the inner cavity 24, which extend in the longitudinal direction of the air channel 20 and are arranged in parallel and adjacent to each other. According to this example, the heat exchange elements 36 are formed as a planar or sheet-like and rectangular shape. However, it is to be understood that the heat exchange elements 36 may have alternative shapes, which are also advantageous for heat transfer. The heat exchange elements 36 provide a significant increase in the surface area at which waste heat can be dissipated and transferred to the ambient air. Due to the orientation of the heat exchange elements 36 the air flow 28 is not substantially impeded or blocked.

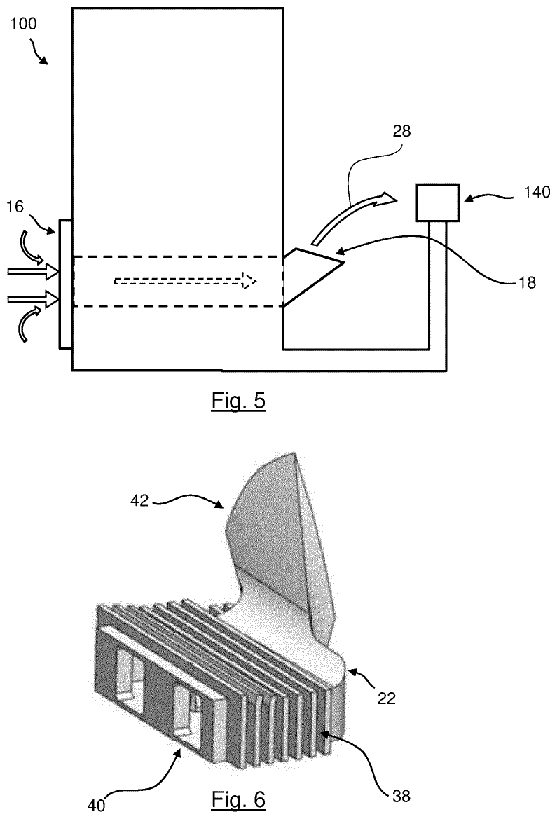

At the air outlet 18, only one opening is provided in the present embodiment (although two or more openings may be provided). Here, the various partial air flows leave or exit the air channel 20 and the cooling device or the medical treatment device in a bundled manner. The air channel 20 is inclined at the air outlet 18 with respect to the upstream portion. Such a design may be advantageous for additional cooling of externally arranged components by means of incident flow and/or impingement cooling (see the following description for FIG. 5).

Furthermore, the surface of the wall 22 facing the interior of the housing also includes a plurality of heat exchange elements 38. However, these extend substantially in the circumferential direction and may be adapted to the air flow 32. That is, the air flow 32 can be provided perpendicularly or transversely to the longitudinal direction of the air channel 20, so as to allow improved heat transfer or dissipation of waste heat to the wall 22.

In FIG. 5, the air flow 28 is shown exiting the air channel 20 at the air outlet 18. According to this embodiment, the inclination of the air channel 20 at the air outlet portion is advantageous for flowing around a corresponding surface of external mechanical components 140, thus enabling additional impingement cooling or film cooling. The mechanical component 140 may be, for example, a pump drive, which can be cooled. Due to the arrangement of the fans and the shape of the air channel 20, efficient cooling of such a pump drive may be provided and thus an advantageous use of the provided air flow 28 can be achieved.

The different configuration of the air channel 20 at the air inlet and at the air outlet with two openings 40 and an inclined opening 42, respectively, is shown in detail in a perspective side view in FIG. 6. However, the exact shape and configuration at the air outlet side or portion may vary and is not limited to the present form.

Furthermore, in this example, it can be seen that the heat exchange elements 38, which are located at the outer surface of the wall 22, extend substantially continuously in the circumferential direction of the air channel 20.

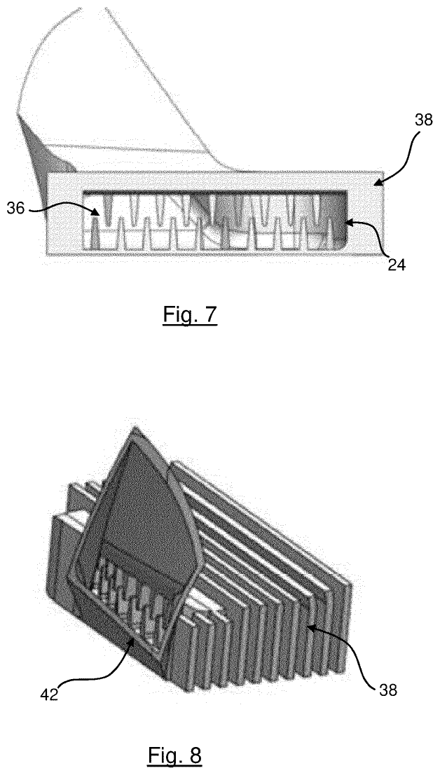

FIGS. 7 and 8 show a perspective sectional view and a perspective rear view, respectively, of the air channel 20 as shown in FIG. 4. From these Figures, an advantageous arrangement of the inner heat exchange elements 36 is shown, wherein the inner heat exchange elements 36 are arranged on opposing sides of the wall 22 in a cross-section of the air channel 20 and wherein the heat exchange elements 36 extending towards each other are arranged offset from each other in the circumferential direction of the air channel 20. As a result, a gap is provided between the adjacent heat exchange elements 36 such that the heat exchange elements 36 do not overlap in any direction perpendicular to the longitudinal direction. In this manner, the surface area provided for dissipating waste heat is further improved without significantly impeding air flow in the air channel by the heat exchange elements 36.

Where applicable, all the individual features depicted in the exemplary embodiments may be combined and/or exchanged without leaving the scope of the invention.

### LIST OF REFERENCE NUMERALS

10 Cooling device
12 Housing
13 Housing wall
14 Housing interior
16 Air inlet
18 Air outlet
20 Air channel
22 Wall
24 Inner cavity
26 Fan
28 Air flow
30 Fan
32 Air flow
34 Electronic component
36 Heat exchange element
38 Heat exchange element
40 Opening
42 Opening
100 Medical treatment device
120 Housing
140 Mechanical component

1-16. (canceled)

17. A cooling device for a medical treatment device, the cooling device comprising:
a housing having an air inlet, an air outlet, and a housing interior defined by a housing wall;
an air channel arranged in the housing interior, wherein a wall of the air channel defines a continuous inner cavity fluidically connected to the air inlet and the air outlet, and wherein the inner cavity, the air inlet, and the air outlet are hermetically sealed from the housing interior by the air channel;
at least one first fan arranged in the inner cavity and adapted to provide a first air flow from the air inlet to the air outlet; and
at least one second fan arranged in the housing interior and adapted to provide a second air flow at least partially flowing around the air channel.

18. The cooling device according to claim 17, wherein the at least one second fan of the housing interior is spaced apart from the wall of the air channel and/or wherein the at least one second fan of the housing interior and the air channel are arranged at opposing end portions of the housing.

19. The cooling device according to claim 17, wherein the wall of the air channel is spaced apart from the housing wall in a portion between the air inlet and the air outlet.

20. The cooling device according to claim 19, wherein the at least one second fan of the housing interior is arranged such that the second air flow flows substantially circumferentially around the wall of the air channel.

21. The cooling device according to claim 17, wherein a portion of the air outlet is configured such that air flow at the air outlet is at least partially inclined relative to the first air flow from the air inlet to the air outlet.

22. The cooling device according to claim 21, wherein an angle of inclination of the air flow at the air outlet relative to the first air flow is 10 degrees to 80 degrees.

23. The cooling device according to claim 17, wherein the at least one first fan comprises two or more fans, wherein the two or more fans are arranged at the air inlet and are equally spaced apart in a longitudinal direction of the air channel with respect to the air inlet.

24. The cooling device according to claim 17, wherein the air channel comprises a plurality of inner heat exchange elements in the inner cavity, wherein the inner heat exchange elements extend substantially in a longitudinal direction of the air channel and protrude from the wall of the air channel into the inner cavity.

25. The cooling device according to claim 24, wherein the inner heat exchange elements are arranged on opposing sides of the wall of the air channel and wherein the inner heat exchange elements extending towards each other are arranged with an offset to each other in a circumferential direction of the air channel.

26. The cooling device according to claim 17, wherein the air channel comprises a plurality of outer heat exchange elements on the wall of the air channel, wherein the plurality of outer heat exchange elements protrude into the housing interior.

27. The cooling device according to claim 26, wherein the plurality of outer heat exchange elements extend circumferentially on the wall of the air channel and are spaced apart from each other in a longitudinal direction.

28. The cooling device according to claim 17, further comprising a nozzle adapted to direct and/or accelerate the first air flow provided in the inner cavity at the air outlet.

29. The cooling device according to claim 28, wherein the nozzle is tiltable relative to the housing wall.

30. The cooling device according to claim 28, wherein a diameter of the nozzle is variable.

31. The cooling device according to claim 17, further comprising a control unit adapted to receive a temperature measurement of the housing interior, the air channel, and/or a medical treatment device cooled by the cooling device, and to control the first air flow provided by the at least one first fan in the air channel based on the temperature measurement.

32. The cooling device according to claim 17, further comprising a control unit adapted to receive an operating condition of a medical treatment device cooled by the cooling device and to control the first air flow provided by the at least one first fan in the air channel based on the operating condition.

33. A medical treatment device comprising:
a cooling device configured to cool the medical treatment device, the cooling device comprising:
a housing having an air inlet, an air outlet, and a housing interior defined by a housing wall,
an air channel arranged in the housing interior, wherein a wall of the air channel defines a continuous inner cavity fluidically connected to the air inlet and the air outlet, and wherein the inner cavity, the air inlet and the air outlet are hermetically sealed from the housing interior by the air channel,
at least one first fan arranged in the inner cavity and adapted to provide a first air flow from the air inlet to the air outlet, and
at least one second fan arranged in the housing interior and adapted to provide a second air flow at least partially flowing around the air channel.

34. The medical treatment device according to claim 33, comprising an extracorporeal blood treatment device and/or extracorporeal circulatory support device.

35. The medical treatment device according to claim 33, further comprising a housing, wherein the housing of the cooling device is integrated in the housing of the medical treatment device, wherein the housing of the cooling device is thermally coupled to the housing of the medical treatment device, or wherein the housing of the cooling device is formed at least as part of the housing of the medical treatment device.

36. The medical treatment device according to claim 33, wherein the at least one second fan of the housing interior is spaced apart from the wall of the air channel and/or wherein the at least one second fan of the housing interior and the air channel are arranged at opposing end portions of the housing.
